(19) United States
(12) Patent Application Publication
(10) Pub. No.: US 2024/0124903 A1
(43) Pub. Date: Apr. 18, 2024

(54) METHODS FOR BIOFUEL AND CHEMICAL PRODUCTION

(71) Applicant: The Regents of the University of California, Oakland, CA (US)

(72) Inventor: Zhiliang Fan, Davis, CA (US)

(73) Assignee: The Regents of the University of California, Oakland, CA (US)

(21) Appl. No.: 18/318,424

(22) Filed: May 16, 2023

Related U.S. Application Data
(60) Provisional application No. 63/344,442, filed on May 20, 2022.

(51) Int. Cl. C12P 7/10; C12P 1/02

(52) U.S. Cl. CPC C12P 7/10; C12P 1/02; C12P 2201/00

(57) ABSTRACT

The present disclosure provides methods for producing fuels, such as biofuels, and commodity chemicals. In some embodiments, the methods comprise pretreating cellulosic biomass with a sugar acid. In some embodiments, the methods further comprise recycling sugar acids that are produced during fermentation for use in the subsequent pretreatment of cellulosic biomass. In some embodiments, the methods further comprise utilizing one or more components produced during pretreatment for subsequent fermentation. Fuels and commodity chemicals produced according to the methods of the present invention are also provided herein.

### CROSS REFERENCE TO RELATED APPLICATIONS

This application claims the benefit of priority from U.S. Provisional Application No. 63/344,442, filed May 20, 2022, titled “METHODS FOR BIOFUEL AND CHEMICAL PRODUCTION,” which is incorporated by reference in its entirety for all purposes.

### BACKGROUND

Low-cost sugar production is the primary driver for adopting biomass feedstocks to produce biofuels and other bio-based chemicals. The biomass production costs vary with feedstock variety, as well as the processing technologies. According to a recent review on sugar costs, the sugar production costs from cellulosic biomass range from $0.10-$3.37/kg, depending on the feedstock types and processing technologies. Feedstock cost contributes to a significant fraction of the sugar cost, followed by pretreatment cost, and cost for the cellulase enzymes.

Cellulosic biorefineries, such as those used in the production of biofuels and various other chemicals, require an effective pretreatment process to facilitate the hydrolysis of cellulosic biomass to fermentable sugars. Existing pretreatment methods include acid or base pretreatment, steam explosion, and ammonia explosion treatment (8,9). During the process of dilute acid pretreatment, hemicellulose is removed from the biomass and degraded to monomeric or oligomeric sugars at elevated temperatures, and lignin is re-adsorbed to cellulose in a modified form (11). While cellulose and hemicellulose hydrolysis increase with increasing severity of the pretreatment conditions, more severe pretreatment conditions also promote the production of various inhibitory compounds. Typically, the choice of pretreatment conditions requires a compromise between sugar yields and degradation product formation (6,10). High pretreatment cost contributes to high processing cost, as does the high cost of cellulase enzymes.

There remains a need in the art for new methods for the production of biofuels and other chemicals. The present disclosure addresses this need, and provides related advantages as well.

### BRIEF SUMMARY

In one aspect, the present disclosure provides a method for producing a fuel or a commodity chemical from a cellulosic biomass. In some embodiments, the method comprises: (a) pretreating a composition comprising the cellulosic biomass with a sugar acid, thereby producing a pretreated composition; (b) fermenting at least some of the pretreated composition in a first fermentation process, thereby producing a first fermented composition, wherein the first fermented composition comprises the sugar acid; (c) separating the first fermented composition into a first fraction and a second fraction, wherein the first fraction comprises at least some of the sugar acid and the second fraction comprises at least some of the sugar acid; and (d) fermenting the second fraction isolated in step (c) in a second fermentation process, thereby producing the fuel and/or commodity chemical.

In some embodiments, the sugar acid comprises an oligosaccharide aldonic acid, a disaccharide aldonic acid, a monosaccharide aldonic acid, a heteropolysaccharide aldonic acid, or a combination thereof. In some embodiments, the sugar acid comprises cellobionic acid (CBA), gluconic acid (GA), glucuronic acid, xylonic acid, glucaric acid, or a combination thereof. In some embodiments, at least some of the sugar acid present in the first fraction separated in step (c) is used in subsequent cellulosic biomass pretreatment.

In some embodiments, the sugar acid is present at a concentration of up to about 99% (e.g., up to about 55%) by volume during the pretreatment in step (a). In some embodiments, the composition being pretreated in step (a) comprises up to about 90% (e.g., up to about 50%; e.g., 0-50%) solids by volume. In some embodiments, the pretreatment in step (a) is performed at a temperature of about 0° C. to about 220° C. (e.g., about 50° C. to about 160° C.). In some embodiments, the pretreatment in step (a) is performed at a pressure of about 0 bar to about 500 bar (e.g., about 0 bar to about 30 bar). In some embodiments, the composition is pretreated in step (a) for about 1 minute to about 2 days (e.g., about 5 minutes to about 2 hours).

In some embodiments, the pretreatment in step (a) is performed in batch, semi-batch, or continuous mode. In some embodiments, the pretreated composition produced in step (a) is separated into a first phase that predominantly comprises liquids and a second phase that predominantly comprises solids. In some embodiments, the first phase comprises hemicellulose hydrolysate, GA, and/or glucose, and at least some of the hemicellulose hydrolysate, GA, and/or glucose are used in the second fermentation process in step (d). In some embodiments, at least some of the GA and/or glucose are produced by hydrolysis of CBA. Further, in some embodiments, the hemicellulose hydrolysate can be further separated into a fraction that contains GA or other aldonic acid, and a fraction that contains sugars using electrodeionization (EDI).

In some embodiments, the second phase comprises pretreated cellulosic biomass, and at least some of the pretreated cellulosic biomass is converted to the sugar acid by an engineered host cell during the first fermentation process in step (b). In some embodiments, the first fermentation process in step (b) comprises an aerobic fermentation process. In some embodiments, the engineered host cell is an engineered fungal cell. In some embodiments, the engineered host cell is an engineered Neurospora crassa cell.

In some embodiments, the engineered host cell comprises: (a) reduced activity of one or more polypeptides having β-glucosidase activity as compared to a corresponding wild-type cell, wherein each of the one or more polypeptides having β-glucosidase activity are encoded by a gene that has at least about 80% sequence identity to a gene selected from the group consisting of NCU00130, NCU04952, NCU05577, NCU07487, NCU08755, and NCU03641; (b) reduced activity of a polypeptide having cellobionate phosphorylase activity as compared to a corresponding wild-type cell, wherein the polypeptide having cellobionate phosphorylase activity is encoded by a gene that has at about least 80% sequence identity to NCU09425 (NdvB); (c) reduced activity of a polypeptide encoded by a gene that has at least about 80% sequence identity to NCU08807 (CRE-1) as compared to a corresponding wild-type cell; and (d) reduced activity of a polypeptide encoded by a gene that has at least about 80% sequence identity to NCU09333 (ACE-1) as compared to a corresponding wild-type host cell.

In some embodiments, the engineered host cell comprises an increased expression or activity of a laccase protein as compared to a corresponding wild-type cell. In certain embodiments, the engineered host cell is cultured in a media that contains cycloheximide to increase the expression or activity of the laccase protein.

In some embodiments, the separation performed in step (c) comprises using electrodeionization (EDI).

In some embodiments, pretreatment of the cellulosic biomass is performed at a lower temperature compared to when the sugar acid is not used to pretreat the cellulosic biomass. In some embodiments, the glucose yield of the pretreatment in step (a) is higher compared to when the sugar acid is not used to pretreat the cellulosic biomass. In some embodiments, the glucose yield of the pretreatment in step (a) is at least about 75% to about 95%. In some embodiments, the amount of cellulase inhibitory compounds that are produced during the pretreatment in step (a) is lower compared to when the sugar acid is not used to pretreat the cellulosic biomass. In some embodiments, the amount of hemicellulose and/or lignin that is removed from the cellulosic biomass during the pretreatment in step (a) is higher compared to when the sugar acid is not used to pretreat the cellulosic biomass.

Moreover, an engineered host cell used in methods of the present disclosure has increased expression or activity of a laccase protein as compared to a corresponding wild-type cell. In some embodiments, a polypeptide having increased laccase expression or activity (i.e., present within an engineered host cell used in methods of the present disclosure) is a polypeptide encoded by NCU04528, which encodes a laccase protein from Neurospora crassa. In some embodiments, the polypeptide having increased laccase expression or activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU04528. Various other laccase proteins are well-known in the art and may be used in the methods and compositions of the present disclosure.

In further embodiments, one or more compounds that increase the amount of the

laccase protein or increase the production or expression of the laccase protein in the engineered host cell can be used. In some embodiments, compounds that increase the amount of the laccase protein or increase the production or expression of the laccase protein can be added exogenously to culture media in methods of the present disclosure. An example of a compound that can increase the amount of the laccase protein or increase the production or expression of the laccase protein is cycloheximide.

In another aspect, the present disclosure provides a fuel and/or a commodity chemical produced according to the methods described herein.

In yet another aspect, the present disclosure provides a method for producing gluconic acid (GA), in which the method comprises separating hemicellulose hydrolysate by electrodeionization (EDI).

Other objects, features, and advantages of the present disclosure will be apparent to one of skill in the art from the following detailed description and figures.

### BRIEF DESCRIPTION OF THE DRAWINGS

FIG. 1 shows the addition of cycloheximide on cellobionate production.

FIG. 2 shows a biochemical route for the production of biofuels and chemicals

according to methods of the present disclosure.

### DETAILED DESCRIPTION

### I. INTRODUCTION

Cellulosic biorefineries require an effective pretreatment process to facilitate the hydrolysis of the cellulosic biomass to fermentable sugars. Leading pretreatment methods include acid or base pretreatment, steam explosion, and ammonia explosion treatment. Among these, dilute acid pretreatment is a promising technology for industrial applications. During the dilute acid pretreatment, hemicellulose is removed from the biomass and degraded to monomeric or oligomeric sugars, and lignin is re-adsorbed to cellulose in a modified form. Sugar yields increase with the increasing severity of the pretreatment conditions, such as longer reaction times, higher acid concentrations, and elevated reaction temperatures. However, more severe pretreatment conditions also promote sugar degradation to inhibitory compounds, such as furfural and 5-hydroxymethylfurfural (HMF). Thus, choosing pretreatment conditions usually involves some compromise between the sugar yield and the formation of degradation products. The acids used for pretreatment are usually consumed during that process. After acid pretreatment, neutralization must be achieved by adding alkali or washing using large quantities of water. All of these contribute to the processing costs.

Organic acid pretreatment using maleic acid, acetic acid, oxalic acid, and succinic acid have been reported in the literature. Compared with dilute inorganic acid pretreatment, organic acid pretreatment has advantages, including effective pretreatment and fewer degradation products (inhibitor formation). However, the organic acids used for pretreatment are often consumed. Neutralization and removal of these acids are also required. Because organic acids are much more expensive than inorganic acids, organic acid pretreatment methods are not cost-competitive with inorganic acid pretreatment methods.

The present disclosure is based, in part, on the discovery of methods for the conversion of cellulosic biomass into biofuels and various other chemicals that comprise pretreating the biomass with a sugar acid. The sugar acids cellobionic acid (CBA), gluconic acid (GA), glucuronic acid, xylonic acid, and glucaric acid, among others, are particularly useful for the pretreatment steps and provide distinct advantages over conventional pretreatment methods including, but not limited to, acid pretreatment. The methods of the present disclosure are particuarly advantageous in that they allow sugar acids to be used as the main hydrolysis product, the main fermentation substrate, and the pretreatment agent. Furthermore, methods of the present disclosure provide economic benefits in that they decrease processing costs and decrease the amounts of various chemical reagents that are required.

### II. DEFINITIONS

It is noted here that as used in this specification and the appended claims, the singular forms “a,” “an,” and “the” include plural reference unless the context clearly dictates otherwise.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “cellulosic biomass” refers to any material that contains cellulose, hemicellulose, and/or lignin, as well as any material that is capable of generating cellulose (e.g., material that produces cellulose as a breakdown product). In some embodiments, cellulosic biomass comprises nonfood biomass. Non-limiting examples are Miscanthus, switchgrass, cord grass, rye grass, reed canary grass, elephant grass, common reed, wheat straw, barley straw, canola straw, oat straw, corn stover, soybean stover, oat hulls, sorghum, rice hulls, rye hulls, wheat hulls, sugarcane bagasse, copra meal, copra pellets, palm kernel meal, corn fiber, Distillers Dried Grains with Solubles (DDGS), Blue Stem, corncobs, pine wood, birch wood, willow wood, aspen wood, poplar wood, energy cane, waste paper, sawdust, forestry wastes, municipal solid waste, waste paper, crop residues, other grasses, other woods, and combinations thereof. Sources of cellulosic biomass can include waste products (e.g., crop or industry waste products) and/or biological material obtained from or derived from dedicated energy crops.

The terms “sugar acid,” “aldonic acid,” and “saccharide aldonic acid” or “SAA” refer to molecules in which the aldehyde functional group of a saccharide has been replaced with a carboxylic acid functional group. Sugar acids can be divided into four general categories: (1) oligosaccharide aldonic acids (OAAs), (2) disaccharide aldonic acids (DAAs), (3) monosaccharide aldonic acids (MAAs), and (4) heteropolysaccharide aldonic acids (HSAAs). Non-limiting examples of OAAs include cellotrionic acid, cellotetraonic acid celloheptonic acid, xylotrionic acid, and xylopentaonic acid. Non-limiting examples of DAAs include cellobionic acid (CBA), lactobionic acid, xylobionic acid, galactonic acid, 4-O-β-D-galactopyranosylgluconic acid, and 6-O-β-D-galactopyranosylgluconic acid. Non-limiting examples of MAAs include gluconic acid, xylonic acid, glucuronic acid, glucaric acid, galactonic acid, arabinic acid, and mannonic acid. An non-limiting example of an HSAA is 4-O-methyl-α-D-glucuronopyranosyl acid.

In the case of DAAs, OAAs, and HSAAs, the connection between sugar units and between the sugar and the end of the aldonic acid can be straight-chain or branched-chain. For example, gluconic acids could be connected glycosidically on the oxygen atoms in the 1-, 3-, 4-, or 6-position of a sugar unit. Furthermore, there can be any combination of sugar and terminal aldonic acid.

In aqueous solution, sugar acids generally exist in salt form. Non-limiting examples of inorganic and organic salts are ammonium, lithium, sodium, magnesium, calcium, and aluminum salts, as well as ethanolamine, triethanolamine, morpholine, pyridine, and piperidine salts.

“Percentage of sequence identity” is determined by comparing two optimally aligned sequences over a comparison window, wherein the portion of the sequence (e.g., a gene sequence or an amino acid sequence present within an engineered cell) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

The term “percent identity” or “percent sequence identity,” in the context of describing two or more polynucleotide or amino acid sequences, refers to two or more sequences or subsequences that are the same or have a specified percentage of amino acid residues or nucleotides that are the same. When a peptide or polynucleotide has at least about 80% sequence identity, preferably at least about 85%, 86%, 87%, 88%, 89%, 90%, 91%, 92%, 93, 94%, 95%, 96%, 97%, 98%, 99%, or 100% identity, to a reference sequence, when compared and aligned for maximum correspondence over a comparison window, or designated region as measured using one of the following sequence comparison algorithms or by manual alignment and visual inspection, such sequences are then said to be “substantially identical.” With regard to polynucleotide sequences, this definition also refers to the complement of a test sequence. Preferably, the identity exists over a region that is at least about 6, 7, or 8 amino acids in length, or more preferably over a region that is at least 6-25 or at least 6-12 amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Additional examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The terms “polypeptide,” “peptide,” and “protein” are used interchangeably herein to refer to a polymer of amino acid residues, or an assembly of multiple polymers of amino acid residues. The terms apply to amino acid polymers in which one or more amino acid residues are artificial chemical mimics of a corresponding naturally occurring amino acid, as well as to naturally occurring amino acid polymers and non-naturally occurring amino acid polymer.

The term “amino acid” includes naturally-occurring a-amino acids and their stereoisomers, as well as unnatural (non-naturally occurring) amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid.

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an α carbon that is bound to a hydrogen, a carboxyl group, an amino group, but have modified R (i.e., side-chain) groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins, 1993).

The term “amino acid modification” refers to a substitution, a deletion, or an insertion of one or more amino acids.

The term “nucleic acid,” “nucleotide” or “polynucleotide” refers to deoxyribonucleic acids (DNA) or ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991), Ohtsuka et al., J. Biol. Chem. 260:2605-2608 (1985), and Rossolini et al., Mol. Cell. Probes 8:91-98 (1994)). The term nucleic acid is used interchangeably with gene, cDNA, and mRNA encoded by a gene.

The term “nucleotide sequence encoding a peptide” or “gene” means the segment of DNA involved in producing a peptide chain, and includes regions preceding and following the coding region (leader and trailer) involved in the transcription/translation of the gene product and the regulation of the transcription/translation, as well as intervening sequences (introns) between individual coding segments (exons).

The term “recombinant” when used with reference, e.g., to a polynucleotide, protein, vector, or cell, indicates that the polynucleotide, protein, vector, or cell has been modified by the introduction of a heterologous nucleic acid or protein or the alteration of a native nucleic acid or protein, or that the cell is derived from a cell so modified. For example, recombinant polynucleotides contain nucleic acid sequences that are not found within the native (non-recombinant) form of the polynucleotide.

### III. DESCRIPTION OF THE EMBODIMENTS

In one aspect, the present disclosure provides a method for producing a fuel (e.g., a biofuel) and/or a commodity chemical from a cellulosic biomass (i.e., a carbon source). In some embodiments, the method comprises: (a) pretreating a composition comprising the cellulosic biomass with a sugar acid, thereby producing a pretreated composition; (b) fermenting at least some of the pretreated composition in a first fermentation process, thereby producing a first fermented composition, wherein the first fermented composition comprises the sugar acid; (c) separating the first fermented composition into a first fraction and a second fraction, wherein the first fraction comprises at least some of the sugar acid and the second fraction comprises at least some of the sugar acid; and (d) fermenting the second fraction isolated in step (c) in a second fermentation process, thereby producing the fuel and/or commodity chemical.

In some embodiments, the sugar acid comprises an oligosaccharide aldonic acid, a disaccharide aldonic acid, a monosaccharide aldonic acid, a heteropolysaccharide aldonic acid, or a combination thereof. In some embodiments, the sugar acid comprises cellobionic acid (CBA), gluconic acid (GA), glucuronic acid, xylonic acid, glucaric acid, or a combination thereof. In some embodiments, at least some of the sugar acid present in the first fraction separated in step (c) is used in subsequent cellulosic biomass pretreatment.

In some embodiments, the sugar acid is cellobionic acid (CBA).

In some embodiments, the sugar acid is gluconic acid (GA). Gluconic acid is a six-carbon multifunctional carbonic acid that has wide application in the construction, chemical, and pharmaceutical industries. It is one of 30 top value-added bio-products derived from sugars identified by the National Renewable Energy Laboratory (NREL). Gluconic acid production is primarily pursued via microbial processes. Microbial production of gluconic acid in submerged culture and solid-state fermentation under aerobic conditions have been widely sought using fungal species, such as Aspergillus niger and Penicillium, and bacterial species, such as Pseudomonas, Acetobacter, and Gluconobacter. Aspergillus niger is the dominant microorganism in the industry. Glucose can be used as the carbon source for microbial production of gluconic acid. Hydrolysate of various raw materials and agro-industrial wastes, such as corn starch, sugarcane molasses, whey, grape must, banana must, and paper waste, have been tested as the alternative substrate to glucose.

In some embodiments, the sugar acid (e.g., CBA, GA, glucuronic acid, xylonic acid, glucaric acid, or a combination thereof) is present at a concentration of up to about 99% (e.g., up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) by volume during the pretreatment in step (a). In some instances, the sugar acid is present at a concentration of up to about 55% (e.g., up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%) by volume.

In some embodiments, the sugar acid (e.g., CBA, GA, glucuronic acid, xylonic acid, glucaric acid, or a combination thereof) is present at a concentration between about 0% and 90%, 0% and 80%, 0% and 70%, 0% and 60%, 0% and 50%, 0% and 40%, 0% and 30%, 0% and 20%, 0% and 10%, 10% and 90%, 10% and 80%, 10% and 70%, 10% and 60%, 10% and 50%, 10% and 40%, 10% and 30%, 10% and 20%, 20% and 90%, 20% and 80%, 20% and 70%, 20% and 60%, 20% and 50%, 20% and 40%, 20% and 30%, 30% and 90%, 30% and 80%, 30% and 70%, 30% and 60%, 30% and 50%, 30% and 40%, 40% and 90%, 40% and 80%, 40% and 70%, 40% and 60%, 40% and 50%, 50% and 90%, 50% and 80%, 50% and 70%, 50% and 60%, 60% and 90%, 60% and 80%, 60% and 70%, 70% and 90%, 70%, and 80%, or 80% and 90% by volume during pretreatment.

In some embodiments, the composition being pretreated in step (a) comprises up to about 90% (e.g., up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%) solids by volume. In some embodiments, the composition being pretreated in step (a) comprises up to about 50% (e.g., up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%) solids by volume.

In some embodiments, the composition being pretreated in step (a) comprises between about 0% and 90%, 0% and 80%, 0% and 70%, 0% and 60%, 0% and 50%, 0% and 40%, 0% and 30%, 0% and 20%, 0% and 10%, 10% and 90%, 10% and 80%, 10% and 70%, 10% and 60%, 10% and 50%, 10% and 40%, 10% and 30%, 10% and 20%, 20% and 90%, 20% and 80%, 20% and 70%, 20% and 60%, 20% and 50%, 20% and 40%, 20% and 30%, 30% and 90%, 30% and 80%, 30% and 70%, 30% and 60%, 30% and 50%, 30% and 40%, 40% and 90%, 40% and 80%, 40% and 70%, 40% and 60%, 40% and 50%, 50% and 90%, 50% and 80%, 50% and 70%, 50% and 60%, 60% and 90%, 60% and 80%, 60% and 70%, 70% and 90%, 70%, and 80%, or 80% and 90% solids by volume

In some embodiments, the pretreatment in step (a) is performed at a temperature of about 0° C. to about 220° C. (e.g., about 0° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., or 220° C.). In some embodiments, the pretreatment in step (a) is performed at a temperature of about 50° C. to about 160° C. (e.g., about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., or 160° C.).

On some embodiments, the pretreatment in step (a) is performed at a temperature of between about 0° C. and 220° C., 0° C. and 210° C., 0° C. and 200° C., 0° C. and 190° C., 0° C. and 180° C., 0° C. and 170° C., 0° C. and 160° C., 0° C. and 150° C., 0° C. and 140° C., 0° C. and 130° C., 0° C. and 120° C., 0° C. and 110° C., 0° C. and 100° C., 0° C. and 90° C., 0° C. and 80° C., 0° C. and 70° C., 0° C. and 60° C., 0° C. and 50° C., 0° C. and 40° C., 0° C. and 30° C., 0° C. and 20° C., 0° C. and 10° C., 10° C. and 220° C., 10° C. and 210° C., 10° C. and 200° C., 10° C. and 190° C., 10° C. and 180° C., 10° C. and 170° C., 10° C. and 160° C., 10° C. and 150° C., 10° C. and 140° C., 10° C. and 130° C., 10° C. and 120° C., 10° C. and 110° C., 10° C. and 100° C., 10° C. and 90° C., 10° C. and 80° C., 10° C. and 70° C., 10° C. and 60° C., 10° C. and 50° C., 10° C. and 40° C., 10° C. and 30° C., 10° C. and 20° C., 20° C. and 220° C., 20° C. and 210° C., 20° C. and 200° C., 20° C. and 190° C., 20° C. and 180° C., 20° C. and 170° C., 20° C. and 160° C., 20° C. and 150° C., 20° C. and 140° C., 20° C. and 130° C., 20° C. and 120° C., 20° C. and 110° C., 20° C. and 100° C., 20° C. and 90° C., 20° C. and 80° C., 20° C. and 70° C., 20° C. and 60° C., 20° C. and 50° C., 20° C. and 40° C., 20° C. and 30° C., 30° C. and 220° C., 30° C. and 210° C., 30° C. and 200° C., 30° C. and 190° C., 30° C. and 180° C., 30° C. and 170° C., 30° C. and 160° C., 30° C. and 150° C., 30° C. and 140° C., 30° C. and 130° C., 30° C. and 120° C., 30° C. and 110° C., 30° C. and 100° C., 30° C. and 90° C., 30° C. and 80° C., 30° C. and 70° C., 30° C. and 60° C., 30° C. and 50° C., 30° C. and 40° C., 40° C. and 220° C., 40° C. and 210° C., 40° C. and 200° C., 40° C. and 190° C., 40° C. and 180° C., 40° C. and 170° C., 40° C. and 160° C., 40° C. and 150° C., 40° C. and 140° C., 40° C. and 130° C., 40° C. and 120° C., 40° C. and 110° C., 40° C. and 100° C., 40° C. and 90° C., 40° C. and 80° C., 40° C. and 70° C., 40° C. and 60° C., 40° C. and 50° C., 50° C. and 220° C., 50° C. and 210° C., 50° C. and 200° C., 50° C. and 190° C., 50° C. and 180° C., 50° C. and 170° C., 50° C. and 160° C., 50° C. and 150° C., 50° C. and 140° C., 50° C. and 130° C., 50° C. and 120° C., 50° C. and 110° C., 50 and 100° C., 50° C. and 90° C., 50° C. and 80° C., 50° C. and 70° C., 50° C. and 60° C., 60° C. and 220° C., 60° C. and 210° C., 60° C. and 200° C., 60° C. and 190° C., 60° C. and 180° C., 60° C. and 170° C., 60° C. and 160° C., 60° C. and 150° C., 60° C. and 140° C., 60° C. and 130° C., 60° C. and 120° C., 60° C. and 110° C., 60° C. and 100° C., 60° C. and 90° C., 60° C. and 80° C., 60° C. and 70° C., 70° C. and 220° C., 70° C. and 210° C., 70° C. and 200° C., 70° C. and 190° C., 70° C. and 180° C., 70° C. and 170° C., 70° C. and 160° C., 70° C. and 150° C., 70° C. and 140° C., 70° C. and 130° C., 70° C. and 120° C., 70° C. and 110° C., 70° C. and 100° C., 70° C. and 90° C., 70° C. and 80° C., 80° C. and 220° C., 80° C. and 210° C., 80° C. and 200° C., 80° C. and 190° C., 80° C. and 180° C., 80° C., and 170° C., 80° C. and 160° C., 80° C. and 150° C., 80° C. and 140° C., 80° C. and 130° C., 80° C. and 120° C., 80° C. and 110° C., 80° C. and 100° C., 80° C. and 90° C., 90° C. and 220° C., 90° C. and 210° C., 90° C. and 200° C., 90° C. and 190° C., 90° C. and 180° C., 90° C. and 170° C., 90° C. and 160° C., 90° C. and 150° C., 90° C. and 140° C., 90° C. and 130° C., 90° C. and 120° C., 90° C. and 110° C., 90° C. and 100° C., 100° C. and 220° C., 100° C. and 210° C., 100° C. and 200° C., 100° C. and 190° C., 100° C. and 180° C., 100° C. and 170° C., 100° C. and 160° C., 100° C. and 150° C., 100° C. and 140° C., 100° C. and 130° C., 100° C. and 120° C., 100° C. and 110° C., 110° C. and 220° C., 110° C. and 210° C., 110° C. and 200° C., 110° C. and 190° C., 110° C. and 180° C., 110° C. and 170° C., 110° C. and 160° C., 110° C. and 150° C., 110° C. and 140° C., 110° C. and 130° C., 110° C. and 120° C., 120° C. and 220° C., 120° C. and 210° C., 120° C. and 200° C., 120° C. and 190° C., 120° C. and 180° C., 120° C. and 170° C., 120° C. and 160° C., 120° C. and 150° C., 120° C. and 140° C., 120° C. and 130° C., 130° C. and 220° C., 130° C. and 210° C., 130° C. and 200° C., 130° C. and 190° C., 130° C. and 180° C., 130° C. and 170° C., 130° C. and 160° C., 130° C. and 150° C., 130° C. and 140° C., 140° C. and 220° C., 140° C. and 210° C., 140° C. and 200° C., 140° C. and 190° C., 140° C. and 180° C., 140° C. and 170° C., 140° C. and 160° C., 140° C. and 150° C., 150° C. and 220° C., 150° C. and 210° C., 150° C. and 200° C., 150° C. and 190° C., 150° C. and 180° C., 150° C. and 170° C., 150° C. and 160° C., 160° C. and 220° C., 160° C. and 210° C., 160° C. and 200° C., 160° C. and 190° C., 160° C. and 180° C., 160° C. and 170° C., 170° C. and 220° C., 170° C. and 210° C., 170° C. and 200° C., 170° C. and 190° C., 170° C. and 180° C., 180° C. and 220° C., 180° C. and 210° C., 180° C. and 200° C., 180° C. and 190° C., 190° C. and 220° C., 190° C. and 210° C., 190° C. and 200° C., 200° C. and 220° C., 200° C. and 210° C., or 210° C. and 220° C.

In some embodiments, the pretreatment in step (a) is performed at a pressure of about 0 bar to about 500 bar (e.g., about 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 bar). In some embodiments, the pretreatment in step (a) is performed at a pressure of about 0 bar to about 30 bar (e.g., about 0, 1, 2, 3, 4, 5, 6, 7, 8, 9, 10, 11, 12, 13, 25 14, 15, 16, 17, 18, 19, 20, 21, 22, 23, 24, 25, 26, 27, 28, 29, or 30 bar).

In some embodiments, the pretreatment in step (a) is performed at a pressure between about 0 bar and 500 bar, 0 bar and 480 bar, 0 bar and 460 bar, 0 bar and 440 bar, 0 bar and 420 bar, 0 bar and 400 bar, 0 bar and 380 bar, 0 bar and 360 bar, 0 bar and 340 bar, 0 bar and 320 bar, 0 bar and 300 bar, 0 bar and 280 bar, 0 bar and 260 bar, 0 bar and 240 bar, 0 bar and 220 bar, 0 bar and 200 bar, 0 bar and 180 bar, 0 bar and 160 bar, 0 bar and 140 bar, 0 bar and 120 bar, 0 bar and 100 bar, 0 bar and 80 bar, 0 bar and 60 bar, 0 bar and 40 bar, 0 bar and 20 bar, 20 bar and 500 bar, 20 bar and 480 bar, 20 bar and 460 bar, 20 bar and 440 bar, 20 bar and 420 bar, 20 bar and 400 bar, 20 bar and 380 bar, 20 bar and 360 bar, 20 bar and 340 bar, 20 bar and 320 bar, 20 bar and 300 bar, 20 bar and 280 bar, 20 bar and 260 bar, 20 bar and 240 bar, 20 bar and 220 bar, 20 bar and 200 bar, 20 bar and 180 bar, 20 bar and 160 bar, 20 bar and 140 bar, 20 bar and 120 bar, 20 bar and 100 bar, 20 bar and 80 bar, 20 bar and 60 bar, 20 bar and 40 bar, 40 bar and 500 bar, 40 bar and 480 bar, 40 bar and 460 bar, 40 bar and 440 bar, 40 bar and 420 bar, 40 bar and 400 bar, 40 bar and 380 bar, 40 bar and 360 bar, 40 bar and 320 bar, 40 bar and 300 bar, 40 bar and 280 bar, 40 bar and 260 bar, 40 bar and 240 bar, 40 bar and 200 bar, 40 bar and 180 bar, 40 bar and 160 bar, 40 bar and 140 bar, 40 bar and 120 bar, 40 bar and 100 bar, 40 bar and 80 bar, 40 bar and 60 bar, 60 bar and 400 bar, 60 bar and 380 bar, 60 bar and 360 bar, 60 bar and 340 bar, 60 bar and 320 bar, 60 bar and 300 bar, 60 bar and 280 bar, 60 bar and 260 bar, 60 bar and 240 bar, 60 bar and 220 bar, 60 bar and 200 bar, 60 bar and 180 bar, 60 bar and 160 bar, 60 bar and 140 bar, 60 bar and 120 bar, 60 bar and 100 bar, 60 bar and 80 bar, 80 bar and 400 bar, 80 bar and 380 bar, 80 bar and 360 bar, 80 bar and 340 bar, 80 bar and 320 bar, 80 bar and 300 bar, 80 bar and 280 bar, 80 bar and 260 bar, 80 bar and 240 bar, 80 bar and 220 bar, 80 bar and 200 bar, 80 bar and 180 bar, 80 bar and 160 bar, 80 bar and 140 bar, 80 bar and 120 bar, 80 bar and 100 bar, 100 bar and 400 bar, 100 bar and 380 bar, 100 bar and 360 bar, 100 bar and 340 bar, 100 bar and 320 bar, 100 bar and 300 bar, 100 bar and 280 bar, 100 bar and 260 bar, 100 bar and 240 bar, 100 bar and 260 bar, 100 bar and 240 bar, 100 bar and 220 bar, 100 bar and 200 bar, 100 bar and 180 bar, 100 bar and 160 bar, 100 bar and 140 bar, 100 bar and 120 bar, 120 bar and 400 bar, 120 bar and 380 bar, 120 bar and 360 bar, 120 bar and 340 bar, 120 bar and 320 bar, 120 bar and 300 bar, 120 bar and 280 bar, 120 bar and 260 bar, 120 bar and 240 bar, 120 bar and 200 bar, 120 bar and 180 bar, 120 bar and 160 bar, 120 bar and 140 bar, 140 bar and 400 bar, 140 bar and 380 bar, 140 bar and 360 bar, 140 bar and 340 bar, 140 bar and 320 bar, 140 bar and 300 bar, 140 bar and 280 bar, 140 bar and 260 bar, 140 bar and 240 bar, 140 bar and 220 bar, 140 bar and 200 bar, 140 bar and 180 bar, 140 bar and 160 bar, 160 bar and 400 bar, 160 bar and 380 bar, 160 bar and 360 bar, 160 bar and 340 bar, 160 bar and 320 bar, 160 bar and 300 bar, 160 bar and 280 bar, 160 bar and 260 bar, 160 bar and 240 bar, 160 bar and 220 bar, 160 bar and 200 bar, 160 bar and 180 bar, 180 bar and 400 bar, 180 bar and 380 bar, 180 bar and 360 bar, 180 bar and 340 bar, 180 bar and 320 bar, 180 bar and 300 bar, 180 bar and 280 bar, 180 bar and 260 bar, 180 bar and 240 bar, 180 bar and 220 bar, 180 bar and 200 bar, 200 bar and 400 bar, 200 bar and 380 bar, 200 bar and 360 bar, 200 bar and 340 bar, 200 bar and 320 bar, 200 bar and 300 bar, 200 bar and 280 bar, 200 bar and 260 bar, 200 bar and 240 bar, 200 bar and 220 bar, 220 bar and 400 bar, 220 bar and 380 bar, 220 bar and 360 bar, 220 bar and 340 bar, 220 bar and 320 bar, 220 bar and 300 bar, 220 bar and 280 bar, 220 bar and 260 bar, 220 bar and 240 bar, 240 bar and 400 bar, 240 bar and 380 bar, 240 bar and 360 bar, 240 bar and 340 bar, 240 bar and 320 bar, 240 bar and 300 bar, 240 bar and 280 bar, 240 bar and 260 bar, 260 bar and 400 bar, 260 bar and 380 bar, 260 bar and 360 bar, 260 bar and 340 bar, 260 bar and 320 bar, 260 bar and 300 bar, 260 bar and 280 bar, 280 bar and 400 bar, 280 bar and 380 bar, 280 bar and 360 bar, 280 bar and 340 bar, 280 bar and 320 bar, 280 bar and 300 bar, 300 bar and 400 bar, 300 bar and 380 bar, 300 bar and 360 bar, 300 bar and 340 bar, 300 bar and 320 bar, 320 bar and 400 bar, 320 bar and 380 bar, 320 bar and 360 bar, 320 bar and 340 bar, 340 bar and 400 bar, 340 bar and 380 bar, 340 bar and 360 bar, 360 bar and 400 bar, 360 bar and 380 bar, or 380 bar and 400 bar.

In some embodiments, the composition is pretreated in step (a) for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. In some embodiments, the composition is pretreated in step (a) for at least about 1, 2, 3, 4, 5, 6, 7, 8, 9, 10, 11, 12, 13, 14, 15, 16, 17, 18, 19, 20, 21, 22, 23, or 24 hours. In some embodiments, the composition is pretreated in step (a) for at least about 0.5, 1, 1.5, 2, 2.5, 3, 3.5, or 4 days. In some embodiments, the composition is pretreated in step (a) for about 1 minute to about 2 days. In some embodiments, the composition is pretreated in step (a) for about 5 minutes to about 2 hours.

In some embodiments, the composition is pretreated in step (a) for between about 10 minutes and 60 minutes, 10 minutes and 50 minutes, 10 minutes and 40 minutes, 10 minutes and 30 minutes, 10 minutes and 20 minutes, 20 minutes and 60 minutes, 20 minutes and 50 minutes, 20 minutes and 40 minutes, 20 minutes and 30 minutes, 30 minutes and 60 minutes, 30 minutes and 50 minutes, 30 minutes and 40 minutes, 40 minutes and 60 minutes, 40 minutes and 50 minutes, 50 minutes and 60 minutes, 2 hours and 24 hours, 2 hours and 22 hours, 2 hours and 20 hours, 2 hours and 18 hours, 2 hours and 16 hours, 2 hours and 14 hours, 2 hours and 12 hours, 2 hours and 10 hours, 2 hours and 8 hours, 2 hours and 6 hours, 2 hours and 4 hours, 4 hours and 24 hours, 4 hours and 22 hours, 4 hours and 20 hours, 4 hours and 18 hours, 4 hours and 16 hours, 4 hours and 14 hours, 4 hours and 12 hours, 4 hours and 10 hours, 4 hours and 8 hours, 4 hours and 6 hours, 6 hours and 24 hours, 6 hours and 22 hours, 6 hours and 20 hours, 6 hours and 18 hours, 6 hours and 16 hours, 6 hours and 14 hours, 6 hours and 12 hours, 6 hours and 10 hours, 6 hours and 8 hours, 8 hours and 24 hours, 8 hours and 22 hours, 8 hours and 20 hours, 8 hours and 18 hours, 8 hours and 16 hours, 8 hours and 14 hours, 8 hours and 12 hours, 8 hours and 10 hours, 10 hours and 24 hours, 10 hours and 22 hours, 10 hours and 20 hours, 10 hours and 18 hours, 10 hours and 16 hours, 10 hours and 14 hours, 10 hours and 12 hours, 12 hours and 24 hours, 12 hours and 22 hours, 12 hours and 20 hours, 12 hours and 18 hours, 12 hours and 16 hours, 12 hours and 14 hours, 14 hours and 24 hours, 14 hours and 22 hours, 14 hours and 20 hours, 14 hours and 18 hours, 14 hours and 16 hours, 16 hours and 24 hours, 16 hours and 22 hours, 16 hours and 20 hours, 16 hours and 18 hours, or 18 hours and 20 hours.

In some embodiments, the composition is pretreated in step (a) for between about 0.2 days and 4 days, 0.2 days and 3.8 days, 0.2 days and 3.6 days, 0.2 days and 3.4 days, 0.2 days and 3.2 days, 0.2 days and 3 days, 0.2 days and 2.8 days, 0.2 days and 2.6 days, 0.2 days and 2.4 days, 0.2 days and 2.2 days, 0.2 days and 2 days, 0.2 days and 1.8 days, 0.2 days and 1.6 days, 0.2 days and 1.4 days, 0.2 days and 1.2 days, 0.2 days and 1 day, 0.2 days and 0.8 days, 0.2 days and 0.6 days, 0.2 days and 0.4 days, 0.4 days and 4 days, 0.4 days and 3.8 days, 0.4 days and 3.6 days, 0.4 days and 3.4 days, 0.4 days and 3.2 days, 0.4 days and 3 days, 0.4 days and 2.8 days, 0.4 days and 2.6 days, 0.4 days and 2.4 days, 0.4 days and 2.2 days, 0.4 days and 2 days, 0.4 days and 1.8 days, 0.4 days and 1.6 days, 0.4 days and 1.4 days, 0.4 days and 1.2 days, 0.4 days and 1 day, 0.4 days and 0.8 days, 0.4 days and 0.6 days, 0.6 days and 4 days, 0.6 days and 3.8 days, 0.6 days and 3.6 days, 0.6 days and 3.4 days, 0.6 days and 3.2 days, 0.6 days and 3 days, 0.6 days and 2.8 days, 0.6 days and 2.6 days, 0.6 days and 2.4 days, 0.6 days and 2.2 days, 0.6 days and 2 days, 0.6 days and 1.8 days, 0.6 days and 1.6 days, 0.6 days and 1.4 days, 0.6 days and 1.2 days, 0.6 days and 1 day, 0.6 days and 0.8 days, 0.8 days and 4 days, 0.8 days and 3.8 days, 0.8 days and 3.6 days, 0.8 days and 3.4 days, 0.8 days and 3.2 days, 0.8 days and 3 days, 0.8 days and 2.8 days, 0.8 days and 2.6 days, 0.8 days and 2.4 days, 0.8 days and 2.2 days, 0.8 days and 2 days, 0.8 days and 1.8 days, 0.8 days and 1.6 days, 0.8 days and 1.4 days, 0.8 days and 1.2 days, 0.8 days and 1 day, 1 day and 4 days, 1 day and 3.8 days, 1 day and 3.6 days, 1 day and 3.4 days, 1 day and 3.2 days, 1 day and 3 days, 1 day and 2.8 days, 1 day and 2.6 days, 1 day and 2.4 days, 1 day and 2.2 days, 1 day and 2 days, 1 day and 1.8 days, 1 day and 1.6 days, 1 day and 1.4 days, 1 day and 1.2 days, 1.2 days and 4 days, 1.2 days and 3.8 days, 1.2 days and 3.6 days, 1.2 days and 3.4 days, 1.2 days and 3.2 days, 1.2 days and 3 days, 1.2 days and 2.8 days, 1.2 days and 2.6 days, 1.2 days and 2.4 days, 1.2 days and 2.2 days, 1.2 days and 2 days, 1.2 days and 1.8 days, 1.2 days and 1.6 days, 1.2 days and 1.4 days, 1.4 days and 4 days, 1.4 days and 3.8 days, 1.4 days and 3.6 days, 1.4 days and 3.4 days, 1.4 days and 3.2 days, 1.4 days and 3 days, 1.4 days and 2.8 days, 1.4 days and 2.6 days, 1.4 days and 2.4 days, 1.4 days and 2.2 days, 1.4 days and 2 days, 1.4 days and 1.8 days, 1.4 days and 1.6 days, 1.6 days and 4 days, 1.6 days and 3.8 days, 1.6 days and 3.6 days, 1.6 days and 3.4 days, 1.6 days and 3.2 days, 1.6 days and 3 days, 1.6 days and 2.8 days, 1.6 days and 2.6 days, 1.6 days and 2.4 days, 1.6 days and 2.2 days, 1.6 days and 2 days, 1.6 days and 1.8 days, 1.8 days and 4 days, 1.8 days and 3.8 days, 1.8 days and 3.6 days, 1.8 days and 3.4 days, 1.8 days and 3.2 days, 1.8 days and 3 days, 1.8 days and 2.8 days, 1.8 days and 2.6 days, 1.8 days and 2.4 days, 1.8 days and 2.2 days, 1.8 days and 2 days, 2 days and 4 days, 2 days and 3.8 days, 2 days and 3.6 days, 2 days and 3.4 days, 2 days and 3.2 days, 2 days and 3 days, 2 days and 2.8 days, 2 days and 2.6 days, 2 days and 2.4 days, 2 days and 2.2 days, 2.2 days and 4 days, 2.2 days and 3.8 days, 2.2 days and 3.6 days, 2.2 days and 3.4 days, 2.2 days and 3.2 days, 2.2 days and 3 days, 2.2 days and 2.8 days, 2.2 days and 2.6 days, 2.2 days and 2.4 days, 2.4 days and 4 days, 2.4 days and 3.8 days, 2.4 days and 3.6 days, 2.4 days and 3.4 days, 2.4 days and 3.2 days, 2.4 days and 3 days, 2.4 days and 2.8 days, 2.4 days and 2.6 days, 2.6 days and 4 days, 2.6 days and 3.8 days, 2.6 days and 3.6 days, 2.6 days and 3.4 days, 2.6 days and 3.2 days, 2.6 days and 3 days, 2.6 days and 2.8 days, 2.8 days and 4 days, 2.8 days and 3.8 days, 2.8 days and 3.6 days, 2.8 days and 3.4 days, 2.8 days and 3.2 days, 2.8 days and 3 days, 3 days and 4 days, 3 days and 3.8 days, 3 days and 3.6 days, 3 days and 3.4 days, 3 days and 3.2 days, 3.2 days and 4 days, 3.2 days and 3.8 days, 3.2 days and 3.6 days, 3.2 days and 3.4 days, 3.4 days and 4 days, 3.4 days and 3.8 days, 3.4 days and 3.6 days, 3.6 days and 4 days, 3.6 days and 3.8 days, or 3.8 days and 4 days hours.

In some embodiments, the pretreatment in step (a) is performed in batch, semi-batch, or continuous mode. In some embodiments, the pretreatment is performed in batch mode. In some embodiments, the pretreatment is performed in semi-batch mode. In some embodiments, the pretreatment is performed in continuous mode.

In some embodiments, the pretreated composition produced in step (a) is separated into a first phase (e.g., that predominantly comprises liquids) and a second phase (e.g., that predominantly comprises solids). In some embodiments, the first phase comprises hemicellulose hydrolysate, GA, and/or glucose. In some embodiments, the first phase comprises hemicellulose hydrolysate. In some embodiments, the first phase comprises GA. In some embodiments, the first phase comprises glucose. The amount of hemicellulose hydrolysate, GA, and/or glucose can vary, e.g., depending on reaction conditions during the pretreatment step such as temperature, time, amount of sugar acid (e.g., CBA, GA, glucuronic acid, xylonic acid, glucaric acid, or a combination thereof) used, etc.

In some embodiments, at least some (e.g., at least about 10%, 20%, 30%, 40%, 50%, 60%, 70%, 80%, or 90%) of the hemicellulose hydrolysate, GA, and/or glucose are used in the second fermentation process in step (d). In some embodiments, essentially all of the hemicellulose hydrolysate, GA, and/or glucose is used in the second fermentation process in step (d). In some embodiments, at least some of the GA and/or glucose is produced by hydrolysis of CBA. The amount of the GA and/or glucose that is produced by CBA hydrolysis can vary, e.g., depending on reaction conditions such as temperature, time, concentration or amount of CBA used, etc.

In some embodiments, the second phase comprises pretreated cellulosic biomass. In some embodiments, at least some (e.g., at least about 10%, 20%, 30%, 40%, 50%, 60%, 70%, 80%, or 90%) of the pretreated cellulosic biomass is converted to the sugar acid (e.g., CBA) by an engineered host cell during the first fermentation process in step (b). In some embodiments, essentially all of the pretreated cellulosic biomass is converted to the sugar acid by an engineered host cell during the first fermentation process in step (b). In some embodiments, the first fermentation process in step (b) comprises an aerobic fermentation process.

Engineered or recombinant host cells used in methods of the present disclosure are capable of producing sugar acids, such as cellobionate, and commodity chemicals from cellulose or cellulosic biomass. The ability to metabolize cellulose is a trait exhibited by lignocellulolytic cells, and thus in some embodiments host cells used in methods of the present disclosure are lignocellulolytic cells. The lignocellulolytic cells may be either aerobic cells or anaerobic cells. In some embodiments, the lignocellulolytic cells are aerobic cells.

The engineered host cells (e.g., lignocellulolytic cells), in some embodiments, produce enzymes that degrade lignocellulose or components thereof. The engineered host cells may degrade the lignocellulose or components thereof under aerobic (i.e., oxygen rich), or anaerobic (i.e., oxygen deficient) conditions. In some embodiments, the engineered host cells are capable of pretreating lignocellulosic biomass. In some embodiments, the engineered host cells simultaneously degrade lignin, solubilize lignin, or change lignin to a revised form, such as demethylized lignin. Lignin is an energy-rich compound that can be utilized for energy production (e.g., electricity). In other embodiments, the engineered host cells produce one or more cellulases, hemicellulases, lignin-solubilizing enzymes, or combinations thereof. In certain embodiments, the one or more hemicellulases and/or lignin-solubilizing enzymes are recombinantly expressed in the engineered host cells. Accordingly, in some embodiments the engineered host cells can produce monosaccharides (e.g., glucose) and cellodextrins (e.g., cellobiose, cellotriose, cellotetrose, cellopentose, etc.) from lignocellulosic biomass. Additionally, in some embodiments the engineered host cells can also produce hemicellulose oligosaccharides, such as xylobiose, from lignocellulosic biomass.

Engineered host cells may be, as non-limiting examples, fungi or bacteria. Suitable fungi for engineering and use in methods of the present disclosure include, but are not limited to, White Rot Fungi, Brown Rot Fungi, Soft Rot Fungi, and ascomycetes fungi. Suitable lignocellulolytic bacteria of the present disclosure may include, for example, Clostridium sp. and Thermanaerobacterium sp. Additional non-limiting examples of suitable host cells include Trichoderma reesei, Clostridium thermocellum, Clostridium papyrosolvens C7, Podospera anserine, Chaetomium globosum, Aspergillus nidulans, Aspergillus niger, Aspergillus oryzae, Saccharomyces cerevisiae, Schizosaccharomyces pombe, Phanerochaete chrysosporium, Sporotrichum thermophile (Myceliophthora thermophila), Gibberella zeae, Sclerotinia sclerotiorum, Botryotinia fuckelian, Aspergillus niger, Thielavia terrestris, Fusarium spp., Rhizopus spp., Neocallimastix frontalis, Orpinomyces sp., Piromyces sp., Penicillium chrysogenum cells, Schizophyllum commune, Postia placenta, Acremonium cellulolyticus, Yarrowia lipolytica, Hansenula polymorpha, Kluyveromyces lactis, Pichia pastoris, Chrysosporium lucknowense, Aspergillus sp., Trichoderma sp., Caldocellulosiruptor sp., Butyrivibrio sp., Butyrivibrio sp., Eubacterium sp., Clostridium sp., Bacteroides sp., Acetivibrio sp., Thermoactinomyces sp., Caldibacillus sp., Bacillus sp., Acidothermus sp., Cellulomonas sp., Micromonospora sp., Actinoplanes sp., Streptomyces sp., Thermobifida sp., Thermomonospora sp., Microbispora sp., Microbispora sp., Fibrobacter sp., Sporocytophaga sp., Cytophaga sp., Flavobacterium sp., Achromobacter sp., Xanthomonas sp., Cellvibrio sp., Pseudomonas sp., Myxobacter sp., Clostridium phytofermentans, Clostridium japonicas, and Thermoanaerobacterium saccharolyticum cells.

In some embodiments, the engineered host cell is a filamentous fungal cell, examples of which include, but are not limited to, Neurospora, Trichoderma, and Aspergillus cells. In some embodiments, the engineered host cell is a Neurospora crassa cell.

Engineered host cells used in methods of the present disclosure are typically living biological cells that are manipulated to alter, for example, the activity of one or more polypeptides in the cell. For example, host cells may be transformed via insertion of recombinant DNA or RNA. Such recombinant DNA or RNA can be in an expression vector. Further, host cells may be subject to mutagenesis to induce mutations in polypeptide-encoding polynucleotides.

In some embodiments, the engineered host cell is modified to facilitate the metabolism of cellulose. As a non-limiting example, the engineered host cell may be modified to contain one or more cellodextrin transporters. Cellodextrins are glucose polymers of varying length and include, for example, cellobiose (2 glucose monomers), cellotriose (3 glucose monomers), cellotetraose (4 glucose monomers), cellopentaose (5 glucose monomers), and cellohexaose (6 glucose monomers). A cellodextrin transporter is any transmembrane protein that transports a cellodextrin molecule from outside of the cell to the inside of the cell and/or from inside of the cell to outside of the cell. Non-limiting examples of suitable cellodextrin transporters include NCU00801, NCU00809, NCU8114, XP_001268541.1, and LAC2.

In some embodiments, an engineered host cell used in methods of the present disclosure has reduced activity of one or more β-glucosidase polypeptides as compared to a corresponding wild-type cell. β-glucosidase (bgl) genes encode β-glucosidase enzymes. As used herein, “β-glucosidase” refers to a β-D-glucoside glucohydrolase (E.C. 3.2.1.21), which catalyzes the hydrolysis of terminal non-reducing β-D-glucose residues with the release of β-D-glucose. A β-glucosidase is any enzyme that catalyzes the hydrolysis of terminal non-reducing residues in β-D-glucosides, such as cellodextrins, with release of glucose. β-glucosidases may be either intracellular β-glucosidases or extracellular β-glucosidases. As used herein, “intracellular β-glucosidases” are expressed within engineered host cells and hydrolyze cellodextrins transported into the cell. As used herein, “extracellular β-glucosidases” are expressed and secreted from engineered host cells or expressed on the surface of lignocellulolytic cells.

In some embodiments, the β-glucosidase is a glycosyl hydrolase family 1 member. Members of this group can be identified by the motif, [LIVMFSTC]-[LIVFYS]-[LIV]-[LIVMST]-E-N-G-[LIVMFAR]-[CSAGN]. Here, E is the catalytic glutamate. In some embodiments, the β-glucosidase is from N. crassa. Other β-glucosidases may include those from the glycosyl hydrolase family 3. These β-glucosidases can be identified by the following motif according to PROSITE: [LIVM](2)-[KR]-x-[EQKRD]-x(4)-G-[LIVMFTC]-[LIVT]-[LIVMF]-[ST]-D-x(2)-[SGADNIT]. Here D is the catalytic aspartate. Typically, any β-glucosidase may be used that contains the conserved domain of β-glucosidase/6-phospho-β-glucosidase/β-galactosidase found in NCBI sequence COG2723.

In certain embodiments, the β-glucosidase is an N. crassa β-glucosidase encoded by NCU00130, NCU04952, NCU05577, NCU07487, NCU08054, NCU08755, and/or NCU03641. Suitable β-glucosidases also include homologs, orthologs, and paralogs of NCU00130, NCU04952, NCU05577, NCU07487, NCU08054, NCU08755, and NCU03641.

Intracellular β-glucosidases include, but are not limited to, those encoded by NCU00130, NCU05577, NCU07487, NCU08054, homologs thereof, orthologs thereof, and paralogs thereof. Extracellular β-glucosidases include, but are not limited to, those encoded by NCU04952, NCU08755, NCU03641, homologs thereof, orthologs thereof, and paralogs thereof.

In some embodiments, the polypeptide having reduced β-glucosidase activity (i.e., in an engineered host cell used in methods of the present invention) is a polypeptide encoded by NCU00130, NCU04952, NCU05577, NCU07487, NCU08755, and/or NCU03641. In some embodiments, the polypeptide having reduced β-glucosidase activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU00130, NCU04952, NCU05577, NCU07487, NCU08755, or NCU03641 genes. Various other (3-glucosidases are well-known in the art and may be used in the methods and compositions of the present disclosure.

One or more β-glucosidases may have reduced activity in engineered host cells used in methods of the present disclosure. Engineered host cells used in methods of the present disclosure may have reduced activity of two or more, three or more, four or more, five or more, or six or more polypeptides having β-glucosidase activity.

In some embodiments, an engineered host cell used in methods of the present disclosure has reduced activity of one or more cellobionate phosphorylase proteins as compared to a corresponding wild-type cell. Cellobionate phosphorylase has activity associated with EC 2.4.1.321 and catalyzes the phosphorolysis of cellobionic acid (4-O-β-D-glucopyranosyl-D-gluconate) to produce α-D-glucose-1-phosphate and D-gluconic acid. In some embodiments, cellobionate phosphorylases used in methods of the present disclosure contain one or more glycosyltransferase family 36 protein domains.

In some embodiments, the polypeptide having reduced cellobionate phosphorylase activity (i.e., present within an engineered host cell used in methods of the present disclosure) is a polypeptide encoded by NCU09425, which encodes the NdvB protein from Neurospora crassa. In some embodiments, the polypeptide having reduced cellobionate phosphorylase activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU09425. Various other cellobionate phosphorylases are well-known in the art and may be used in the methods of the present disclosure. Other exemplary cellobionate phosphorylases include, for example, a glycoside hydrolase family 94 cellobionate phosphorylase from the bacterium Xanthomonas campestris.

In some embodiments, an engineered host cell used in methods of the present disclosure has reduced activity of a CRE-1 protein as compared to a corresponding wild-type cell. CRE-1 is a transcription factor protein. Without being bound by any particular theory, it is thought that CRE-1 is involved in catabolite repression. Deletion of the cre has been shown to increase cellulase expression. A cre-strain has decreased growth rate on preferred carbon sources; however, on Avicel it produces 30-50% more cellulase and consumes avicel faster, and cre expression correlates with cellulase expression.

In some embodiments, the polypeptide having reduced CRE-1 activity (i.e., present within an engineered host cell used in methods of the present disclosure) is a polypeptide encoded by NCU08807, which encodes a CRE-1 protein from Neurospora crassa. In some embodiments, the polypeptide having reduced CRE-1 activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU08807. Various other CRE-1 proteins are well-known in the art and may be used in the methods and compositions of the present disclosure.

In some embodiments, an engineered host cell used in methods of the present disclosure has reduced activity of an ACE-1 protein as compared to a corresponding wild-type cell. ACE-1 is a zinc finger transcription factor protein. Mutation of the ace-1 gene (ace-) results in higher cellulase expression in T. reesei.

In some embodiments, the polypeptide having reduced ACE-1 activity (i.e., present within an engineered host cell used in methods of the present disclosure) is a polypeptide encoded by NCU09333, which encodes an ACE-1 protein from Neurospora crassa. In some embodiments, the polypeptide having reduced ACE-1 activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU09333. Various other ACE-1 proteins are well-known in the art and may be used in the methods and compositions of the present disclosure.

In some embodiments, an engineered host cell used in methods of the present disclosure has reduced activity of a MUSS 1 protein as compared to a corresponding wild-type cell. MUSS 1 is an ATP-dependent DNA helicase II subunit 1 protein (E.C. 3.6.4.12). MUSS 1 proteins are involved in non-homologous end joining (NHEJ) DNA double strand break repair. Without being bound by any particular theory, it is thought that reducing MUSS 1 protein activity in a host cell will increase the chances for and/or frequency of homologous recombination in the host cell, which may make genetic modification of the host cell easier.

In some embodiments, the polypeptide having reduced MUSS 1 activity (i.e., present within an engineered host cell used in methods of the present disclosure) is a polypeptide encoded by NCU08290, which encodes a MUSS 1 protein from Neurospora crassa. In some embodiments, the polypeptide having reduced MUSS 1 activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU08290. Various other MUS51 proteins are well-known in the art and may be used in the methods of the present disclosure.

In some embodiments, an engineered host cell used in methods of the present disclosure has increased expression or activity of a laccase protein as compared to a corresponding wild-type cell. Laccase proteins may also be added exogenously to culture media in methods of the present disclosure. Laccases are “blue” copper containing oxidases (E.C. 1.10.3.2). Laccases catalyze the following reaction: 4 benzenediol+O₂=4 benzosemiquinone+2H₂O. These multi-copper enzymes have low specificity acting on both o- and p-quinols, and often act also on aminophenols and phenylenediamine. Laccases may also act on phenols and similar molecules, performing one-electron oxidations. Because laccase proteins belong to the oxidase enzyme family, these enzymes require oxygen as a second substrate for enzymatic action.

In some embodiments, the polypeptide having increased laccase expression or activity (i.e., present within an engineered host cell used in methods of the present disclosure) is a polypeptide encoded by NCU04528, which encodes a laccase protein from Neurospora crassa. In some embodiments, the polypeptide having increased laccase expression or activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU04528. Various other laccase proteins are well-known in the art and may be used in the methods and compositions of the present disclosure.

In further embodiments, one or more compounds that increase the amount of the laccase protein or increase the production or expression of the laccase protein in the engineered host cell can be used. In some embodiments, compounds that increase the amount of the laccase protein or increase the production or expression of the laccase protein can be added exogenously to culture media in methods of the present disclosure. An example of a compound that can increase the amount of the laccase protein or increase the production or expression of the laccase protein is cycloheximide.

In some embodiments, an engineered host cell used in methods of the present disclosure has increased expression or activity of a cellobiose dehydrogenase protein as compared to a corresponding wild-type cell. Cellobiose dehydrogenase proteins may also be added exogenously to culture media in methods of the present disclosure. Cellobiose dehydrogenase (CDH) enzymes catalyze the following reaction: cellobiose+acceptor=cellobiono-1,5-lactone+reduced acceptor (E.C. 1.1.99.18). CDH proteins contain an N-terminal heme domain and a C-terminal dehydrogenase domain. Some CDH proteins also contain a cellulose binding module (CBM) at the C-terminus of the protein. Orthologs of the CDH heme domain are found only in fungal proteins, whereas orthologs of the dehydrogenase domain are found in proteins throughout all domains of life; the dehydrogenase domain is part of the larger GMC oxidoreductase superfamily.

In some embodiments, the polypeptide having increased cellobiose dehydrogenase expression or activity (i.e., present within an engineered host cell used in methods of the present disclosure) is a polypeptide encoded by NCU00206, which encodes a cellobiose dehydrogenase protein from Neurospora crassa. In some embodiments, the polypeptide having increased cellobiose dehydrogenase expression or activity is a polypeptide encoded by a gene that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to NCU00206. Various other cellobiose dehydrogenase proteins are well-known in the art and may be used in the methods and compositions of the present disclosure. Other cellobiose dehydrogenases include, for example, the polypeptides of Accession Numbers: XM_411367, BAD32781, BAC20641, XM_389621, AF257654, AB187223, XM_360402, U46081, AF081574, AY187232, AF074951, and AF029668.

In some embodiments, the engineered host cell comprises: (a) reduced activity of one or more polypeptides having β-glucosidase activity as compared to a corresponding wild-type cell, wherein each of the one or more polypeptides having β-glucosidase activity are encoded by a gene that has at least about 80% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a gene selected from the group consisting of NCU00130, NCU04952, NCU05577, NCU07487, NCU08755, and NCU03641; (b) reduced activity of a polypeptide having cellobionate phosphorylase activity as compared to a corresponding wild-type cell, wherein the polypeptide having cellobionate phosphorylase activity is encoded by a gene that has at about least 80% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to NCU09425 (NdvB); (c) reduced activity of a polypeptide encoded by a gene that has at least about 80% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to NCU08807 (CRE-1) as compared to a corresponding wild-type cell; and (d) reduced activity of a polypeptide encoded by a gene that has at least about 80% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to NCU09333 (ACE-1) as compared to a corresponding wild-type host cell. Engineered host cells that can be used in methods of the present disclosure are also described further in U.S. Patent Application Publication No. US 2017/0044583, which is hereby incorporated in its entirety for all purposes.

For separating the first fermented composition, any suitable method can be used. In some embodiments, the separation performed in step (c) comprises using electrodeionization (EDI). EDI is a water treatment technology that uses electricity, ion exchange membranes, and resins in the deionization of water, thus separating impurities from water molecules. In some embodiments, the first fermented composition is separated into two or more (e.g., 2, 3, 4, 5, or more) fractions. In some embodiments, the first fermented composition is separated into 2 fractions (i.e., a first fraction and a second fraction).

In some embodiments, pretreatment of the cellulosic biomass is performed at a lower temperature compared to when the sugar acid (e.g., CBA, GA, glucuronic acid, xylonic acid, glucaric acid, or a combination thereof) is not used to pretreat the cellulosic biomass. In some embodiments, pretreatment is performed at a temperature that is at least about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.5-fold, 6-fold, 6.5-fold, 7-fold, 7.5-fold, 8-fold, 8.5-fold, 9-fold, 9.5-fold, or 10-fold lower than the temperature that is used when the sugar acid is not used for the pretreatment. In some embodiments, pretreatment is performed at a temperature that is at least about 10° C., 20° C., 30° C., 40° C., 50° C., 60° C., 70° C., 80° C., 90° C., 100° C., 110° C., 120° C., 130° C., 140° C., 150° C., or 160° C. lower than the temperature that is used when the sugar acid is not used for the pretreatment.

In some embodiments, a sugar (e.g., glucose) yield of the pretreatment in step (a) is higher compared to when the sugar acid (e.g., CBA, GA, glucuronic acid, xylonic acid, glucaric acid, or a combination thereof) is not used to pretreat the cellulosic biomass. In some embodiments, the sugar (e.g., glucose) yield of the pretreatment step is at least about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.5-fold, 6-fold, 6.5-fold, 7-fold, 7.5-fold, 8-fold, 8.5-fold, 9-fold, 9.5-fold, or 10-fold higher compared to when the sugar acid (e.g., CBA, GA, glucuronic acid, xylonic acid, glucaric acid, or a combination thereof) is not used to treat the cellulosic biomass. In some embodiments, the sugar (e.g., glucose) yield of the pretreatment in step (a) is at least about 80%, 81%, 82%, 83%, 84%, 85%, 86%, 87%, 88%, 89%, 90%, 91%, 92%, 93%, 94%, 95%, 96%, 97%, 98%, or 99%. In particular embodiments, the sugar (e.g., glucose) yield is between about 85% and 95%.

In some embodiments, the amount of cellulase inhibitory compounds (e.g., furfural, 5-hydroxymethylfurfural (HMF), or formic acid) that are produced during the pretreatment in step (a) is lower compared to when the sugar acid (e.g., CBA, GA, glucuronic acid, xylonic acid, glucaric acid, or a combination thereof) is not used to pretreat the cellulosic biomass. In some embodiments, the amount of cellulase inhibitor compounds that are produced during pretreatment is at least about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.5-fold, 6-fold, 6.5-fold, 7-fold, 7.5-fold, 8-fold, 8.5-fold, 9-fold, 9.5-fold, or 10-fold lower compared to when the sugar acid is not used to pretreat the cellulosic biomass.

In some embodiments, the amount of hemicellulose and/or lignin that is removed from the cellulosic biomass during the pretreatment in step (a) is higher compared to when the sugar acid (e.g., CBA, GA, glucuronic acid, xylonic acid, glucaric acid, or a combination thereof) is not used to pretreat the cellulosic biomass. In some embodiments, the amount of hemicellulose that is removed from the cellulosic biomass during pretreatment is at least about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.5-fold, 6-fold, 6.5-fold, 7-fold, 7.5-fold, 8-fold, 8.5-fold, 9-fold, 9.5-fold, or 10-fold higher compared to when the sugar acid is not used to pretreat the cellulosic biomass. In some embodiments, the amount of lignin that is removed from the cellulosic biomass during pretreatment is at least about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.5-fold, 6-fold, 6.5-fold, 7-fold, 7.5-fold, 8-fold, 8.5-fold, 9-fold, 9.5-fold, or 10-fold higher compared to when the sugar acid is not used to pretreat the cellulosic biomass.

In another aspect, the present disclosure provides a fuel (e.g., a biofuel) and/or a commodity chemical produced according to the methods described herein. In some embodiments, the fuel is an ethanol fuel. Non-limiting examples of commodity chemicals include, alcohol, ethanol, propanol, isopropanol, acetone, butanol, isobutanol, 2-methyl-1-butanol, 3-methyl-1-butanol, phenylethanol, a fatty alcohol, isopentenol, an aldehyde, an acetylaldehyde, a propionaldehyde, a butryaldehyde, an isobutyraldehyde, 2-methyl-1-butanal, 3-methyl-1-butanal, phenylacetaldehyde, a fatty aldehyde, a hydrocarbon, an alkane, an alkene, an isoprenoid, a fatty acid, a wax ester, an ethyl ester, hydrogen, and combinations thereof.

### IV. EXAMPLES

The present disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Sugar Yield of Pretreatment with Gluconic Acid and Cellobionic Acid as Pretreatment Agent

Wheat straw was milled to the particle size of 0.5 to 1 mm. Pretreatment was performed in a 14 mL sealed tube reactor with a working volume of 9 mL in an oil bath. Critical parameters include, e.g., temperature (170-190° C.), acid concentrations (125-1000 mM), and pretreatment duration (controlled at 30 minutes). The pretreatment reactions were be terminated by immersing the reactor in ice water until the internal temperature decreases below 50° C.

Vacuum filtration was conducted to separate the resulting solids from the liquid. A small portion of filtrate was be neutralized with CaCO₃ and filtered using glass filter paper with a pore size of 0.20 μm prior to sugar analysis. Another portion of the filtrate was collected for analysis of degradation products (acetic acid, GA, furfural, and HMF). Oligomers were analyzed by hydrolyzing in 4% sulfuric acid at 121° C. for 1 hour. The resulting solids were washed by large amounts of water and their composition determined following an NREL protocol. The corresponding glucan, xylan, lignin, glucose, and xylose yields were calculated.

The washed solids were enzymatically hydrolyzed using commercial cellulase preparations with and without the addition of β-glucosidase to determine sugar yields obtained from different pretreatment conditions. In addition, to establish baseline performance, how sugar release from the pretreated cellulosic feedstock varies with enzyme loading of 60 FPU/g of cellulose was explored.

Table 1 below shows sugar yield of pretreatment using gluconic acid or cellobionic acid as pretreatment agent.

TABLE 1
Comparison of the conventional platform and
the sugar aldonic acid-based refinery

| | Concentration of gluconic acid pretreatment | | | |
|---|---|---|---|---|
| | 0.125M | 0.25M | 0.5M | 1M |
| Yield at 170° C. | 73.6000% | 76.5000% | 82.5000% | 78.7000% |
| Yield at 180° C. | 73.7000% | 72.1000% | 77.3000% | 78.0000% |
| Yield at 190° C. | 70.3000% | 67.7000% | 72.3000% | 64.2000% |

| | Concentration of cellobionic acid pretreatment |
|---|---|
| | 0.338M |
| Yield at 160° C. | 82% |

### Example 2. Addition of Cycloheximide Promoting CBA Production by Inducing Laccase Production

The strain HL10 was grown on alkaline pretreated wheat straw as the carbon source in 1× Vogel's media, 3 g/L glucose, and 0.8 mM of CuSO₄. The initial wheat straw amount was normalized to 20 g/L cellulose equivalent. Cycloheximide (CH) was added at 0.3 μM on day 2. Samples were withdrawn from the flasks to measure cellobionate production.

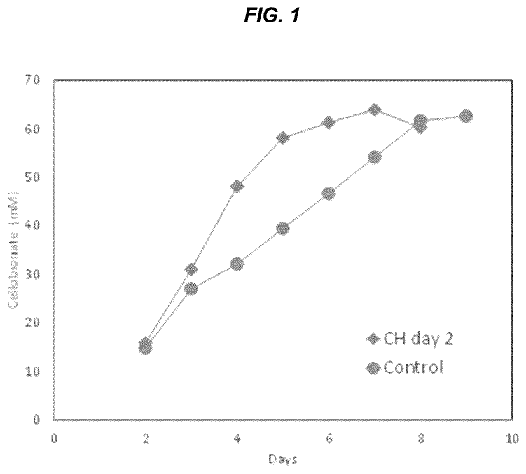

As shown in FIG. 1, the cellobionate production reached the highest level of production earlier as compared to without cycloheximide addition.

### Example 3. Sugar Aldonic Acid-Based Biorefinery

### Concept Summary

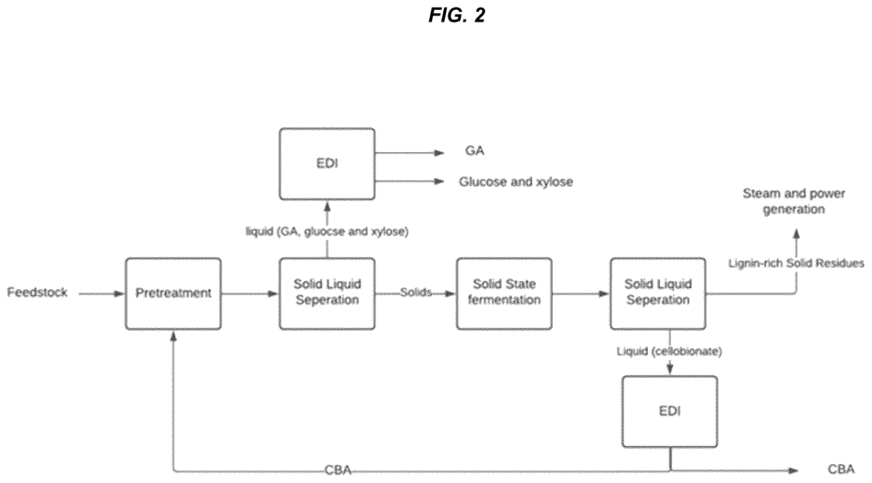

In conventional biological platforms for fuel and chemical production from cellulosic biomass, sugars are usually produced as a hydrolysis product and serve as a fermentation substrate for further conversion to biofuels. As described herein, the present disclosure relates to a sugar aldonic acid based biorefinery concept, in which cellobionic acid (CBA) serves as the main hydrolysis product, the main fermentation substrate, and the pretreatment agent. As illustrated in FIG. 2, the feedstock undergoes a CBA pretreatment step first. The pretreated solids are then converted to CBA by an engineered fungal strain through aerobic fermentation without any enzyme addition. The resulting CBA is then recovered through a separation process such as electrodeionization (EDI) and used as the substrate for subsequent fermentation to produce fuels and chemicals, as well as for subsequent use as the pretreatment agent. During the pretreatment process, CBA is hydrolyzed to glucose and gluconic acid (GA) in the hemicellulose hydrolysate stream, both of which are used as substrates for subsequent fermentation. This disclosure is particularly advantageous in that it lowers the processing cost of cellulosic biomass conversion to produce renewable biofuels.

### Innovation and Impact

The present disclosure possesses several novel features and advantages, some of which are described below.

### Novel Pretreatment Agent and Process

High pretreatment cost is one of the major contributors to high processing cost. As an organic acid of pKₐ 3.4, CBA is able to remove hemicellulose like other organic or inorganic acids. During the process of CBA pretreatment, CBA is hydrolyzed into glucose and GA (pKₐ 3.8), both of which then serve as fermentation substrates and get recycled in the subsequent fermentation process. It is not economically feasible to use high catalyst concentrations in an acid pretreatment process if the acid used is not recycled during the process, although higher catalyst concentrations favor faster hydrolysis and reduced inhibitor formation. Since CBA is fully recyclable, pretreatment can be carried out at higher acid concentrations and lower temperatures, reducing processing costs, lowering inhibitor formation, and improving pretreatment selectivity. The pretreatment also serves the purpose of producing GA from CBA. CBA pretreatment is essentially GA pretreatment due to CBA hydrolysis during the pretreatment process. Both GA and CBA are organic solvents, which have the ability to dissolve not only hemicellulose, but also lignin, leading to higher cellulose digestibility and sugar yields.

### Novel Process Intensification Combining Cellulase Production and Hydrolysis Into One Step

The high cost of cellulase enzymes is another major contributor to high processing cost. Cellulase enzymes are produced by cellulolytic fungi via aerobic fermentation. If a cellulolytic fungus is used to achieve direct cellulase production and hydrolysis, the cellulolytic fungus will inevitably consume the resulting sugars during the process of hydrolysis, which leads to severe carbon loss. In addition, the inhibition of cellulases by the produced sugars limits the sugar titers and the process productivity. The present disclosure provides a novel strategy to overcome this problem: use of a cellulolytic fungus that has been modified to hydrolyze cellulose and produce cellobionate as the main hydrolysis product.

Hence it can be produced at higher titers. Cellobionate is less inhibitory to cellulase than sugars and cannot be consumed by the fungus.

Starting from the wild-type strain N. crassa 2489, which produces a full array of cellulases, genes encoding enzymes for cellobiose or cellobionate utilization were disabled. With the presence of cellobiose dehydrogenase (CDH) naturally produced by the fungus and a heterologously expressed laccase that facilitates the electron transfer from cellobiose to oxygen with the help of a redox mediator, cellobiose was dehydrogenized to cellobionate. Cellobionate can be produced from cellulose at a yield of 95% from the consumed cellulose with redox mediator addition. When the cellulosic substrate is used as the substrate, lignin and lignin degradation products contained in lignocellulosic biomass could naturally serve as redox mediators. Thus, no exogenous redox mediator is needed for the conversion. The engineered Neurospora crass strain HL10 produced CBA from pretreated wheat straw at a yield of 85% (based on starting cellulose) in 10 days without any exogenous enzyme addition via solid-state fermentation. The titer was about 44 g/L.

CBA and GA are charged molecules, which facilitate their separation from the fermentation broth or hemicellulose hydrolysate via an environmentally friendly and chemical-free electrodeionization (EDI) process. EDI is an industrial hybrid technology that combines electrodialysis and ion-exchange resin column (IEC) processes into a single operation. It is used commercially to produce ultrapure water for the semiconductor and pharmaceutical industries. Resin-wafer EDI (RW-EDI) technology was developed at the Argonne National Laboratory (ANL). In RW-EDI, loose ion exchange resins are molded into a solid, porous resin wafer, which increases ionic conductivity, porosity, and performance consistency in comparison to a loose resin bed. RW-EDI improves pH control, process stream control, and ion management and enables its application to recover organic acids from fermentation broth. The ion resin beads serve as an ion reservoir in the feed compartment that both drive weak organic acid dissociation into organic ions and increase organic ion concentrations by at least two orders of magnitude. The ions in the high concentration reservoir (resin wafer beads) are electrically transported across the ion-exchange membrane. The significant increase in solution conductivity by the ion reservoir enables high-efficiency separation of organic acids by EDI. Besides, bases will be generated along with the acid from the EDI process, which can be used for fermentation pH control. The cost of RW-EDI for organic acid recovery is relatively insensitive to the feed organic acid concentration.

EDI has been used to recover lactic acid, tartaric acid, and citric acid from fermentation broth. Specifically, RW-EDI technology has separated butyric acid from acetic acid in the fermentation broth in a continuous fermentation process. It was also used to couple with enzymatic and fermentative gluconic acid production and produced pure gluconic acid with a separation efficiency of 99%.

The integration of a highly efficient acid separation technology (EDI) with the aerobic fermentation can significantly facilitate the bioconversion by in-situ capture of CBA once it is produced in the fermentation broth. Such an integrated aerobic fermentation and EDI separation process, called a separative bioreactor, has been demonstrated by pilot-scale operation in an industrial site and proved to be technically and economically viable. The innovative separative bioreactor produces a concentrated CBA product stream, which is directly used for both pretreatment and subsequent fermentation (4).

### Superior Substrate for Aerobic Fermentation for Triacylglycerol (TAG) and Polyhydroxyalkanoate (PHA) Production

Although sugars are universal substrates for microbial growth, sugar aldonic acids

(aldonates) have some advantages over sugars in aerobic processes pertaining to triacylglycerol and PHA production by actinomycetes bacteria. For example, the bacterium Rhodococcus opacus PD630 accumulates up to 80% of its cell dry weight as TAG when gluconate was used as the substrate, as compared to about 50% when glucose is used as the substrate (1). Gluconate is used even faster than that of glucose.

Some of the advantages of the present disclosure over conventional methods are shown below in Table 2.

TABLE 2
Comparison of the conventional platform and the sugar aldonic acid-based refinery

| Conventional Biorefinery | Sugar acid based Biorefinery |
|---|---|
| Use inorganic acid or organic | Use CBA as the pretreatment agent. CBA is hydrolyzed into |
| acid as the pretreatment agent. | glucose and GA during the pretreatment process, recovered |
| Acid recycling is highly desired | as fermentation substrates. The advantages of CBA |
| but cost-effectiveness is a | pretreatment include the in situ production of the |
| challenge. For this reason, | pretreatment agent, easy recycling of the pretreatment agent, |
| diluted acid and high temperate | potentially decreased inhibitor formation and increased |
| are usually preferred. Acid used | cellulose digestibility due to lignin removal in the |
| in the process needs to be | pretreatment step, and full compatibility of CBA |
| neutralized or removed. | pretreatment with the subsequent fermentation process. |
| Aerobic fermentation to produce | Combine the cellulase production and enzymatic hydrolysis |
| cellulase. | into one single aerobic fermentation step. Using an |
| Cellulase enzymes are added to | engineered fungus strain to produce CBA instead of sugar as |
| pretreated cellulosic biomass to | hydrolysis products without exogenous adding cellulase |
| achieve hydrolysis. The end | enzymes. CBA, as a charged molecule, is much easier to be |
| product cellobiose and glucose | separated and concentrated from the fermentation broth than |
| inhibit the cellulase enzyme. | sugars. |
| Their removal is difficult. | |
| Using sugars as the fermentation | Use gluconate or cellobionate in addition to hemicellulose |
| substrate. TAG can be | sugars as the substrate. R. opacus PD630 can accumulate up |
| accumulated up to 50% when | to 80% TAG as cellular dry weight when gluconate is used |
| glucose is used as the substrate. | as substrate; and it uses gluconate faster than glucose. |

### Rationale and Significance

### Rationale

Cellulosic biorefineries require an effective pretreatment process to facilitate the hydrolysis of cellulosic biomass to fermentable sugars. Leading pretreatment methods include acid or base pretreatment, steam explosion, and ammonia explosion treatment (8,9). Among these, dilute acid pretreatment is a promising technology for industrial applications. During the process of dilute acid pretreatment, hemicellulose is removed from the biomass and degraded to monomeric or oligomeric sugars at elevated temperatures, and lignin is re-adsorbed to cellulose in a modified form (11). The cellulose and hemicellulose hydrolysis increase with increasing severity of the pretreatment conditions, such as longer reaction time, higher acid concentrations, and elevated reaction temperatures. However, more severe pretreatment conditions also promote sugar degradation to inhibitory compounds such as furfural, 5-hydroxymethylfurfural (HMF), and formic acid. The choice of pretreatment conditions usually requires a compromise between sugar yields and degradation product formation (6,10).

The acids used for pretreatment are generally consumed during the process of pretreatment. Alkali chemicals are needed for neutralization, or large quantities of water are needed for washing after the acid pretreatment. All of these requirements contribute to processing costs (8,9).

According to a kinetic study of maleic acid pretreatment, higher acid concentrations and lower temperatures promote the selectivity of the acid pretreatment (5), which is defined as the ratio of the reaction rate constant of the hydrolysis reaction to the reaction rate constant of the degradation reaction (7). However, it is not economically feasible to use high catalyst concentrations in an acid pretreatment process if the acid used is not recycled during the process (5).

In stark contrast to conventional methods, the present disclosure uses CBA as a pretreatment agent. CBA is a weak organic acid with a pKₐ of approximately 3.28. CBA pretreatment has advantages including, but not limited to, low inhibitor formation to other dilute organic acid pretreatment methods. An additional advantage of CBA pretreatment is its non-toxicity to microbial fermentation. Neutralization generates cellobionate, which instead of being used as waste, can be used as a substrate for microbial fermentation in the subsequent process. In some embodiments, the pretreatment, CBA production, and recycling are integrated. After pretreatment, the small amount of CB A remaining in the solids is carried over to fermentation and recycled with the CBA produced at the end of fermentation. The liquid fraction contains both CBA and hemicellulose sugars, which are substrates for the subsequent fermentation. The full recyclability of CBA as a pretreatment agent allows it to be used in higher concentrations (e.g., 50%) and lower temperature (e.g., 100-130° C.). As indicated in an acid pretreatment kinetic study, the higher acid concentration at lower temperatures leads to higher sugar yields, reduced inhibitor formation, and lower processing cost.

Various studies have reported that enzymatic digestibility of pretreated solids improves with increasing lignin removal (12). In the process of diluted inorganic acid pretreatment, the lignin is depolymerized during the process of acid pretreatment (11). The lignin removal by dilute acid pretreatment in batch reaction is very limited. As an organic acid and a solvent, CBA or GA, when used at higher concentrations, can dissolve both hemicellulose and lignin. Thus, improved lignin removal improves overall sugar yields.

### Preliminary Comparison of the CBA Pretreatment and the Diluted Acid Pretreatment

The analysis summarized in Table 3 below (i.e., dollar savings per metric ton of biomass) assumes cellulose content of 35% and solid loading of 30%. Diluted acid pretreatment data was based on the 2011 National Renewable Energy Laboratory (NREL) process design (2). The diluted sulfuric acid is used at the concentration of 1.8%. The CBA is assumed to be used with the concentration of 30%. Same reaction time is assumed.

TABLE 3
Comparison of CBA pretreatment to diluted acid pretreatment

| Diluted acid pretreatment | CBA pretreatment | Saving ($ per ton of biomass) |
|---|---|---|
| Reaction in 160° C. | Reaction in 110° C. (lower | 239 kg less high pressure |
| | reaction temperature leads to | steam will be used, |
| | energy savings. | assuming the high pressure |
| | | steam cost $16.4/ton |
| | | Energy saving is about $3.89 |
| Use 3.3 ton of 1.8% H₂SO₄ | No need to add additional | Sulfuric acid ($81.38/ton) |
| | acid | acid consumption will cost |
| | | about $4.83 |
| Use ammonia to neutralize | No need to add additional | Ammonia ($407/ton) |
| acid | base to neutralize the | consumption will cost $3.62 |
| | additional acid added; the | |
| | base to neutralize CBA will | |
| | be generated during the | |
| | subsequent EDI process | |
| Glucose yield 85% | Glucose yield 95% (higher | Extra 35 kg of glucose can be |
| | sugar yields due to lignin | produced, assuming sugar |
| | removal) | cost of $0.6/kg, extra sugar |
| | | produced will worth about |
| | | $21 |
| More inhibitor formation | Less inhibitor formation, | N/A |
| | higher quality fermentation | |
| | substrate for subsequent | |
| | fermentation | |

In summary, some of the key features of the present disclosure are:
1) Sugar aldonic acids such as CBA and GA are used at a concentration of about 0-99%. The acid solution and the material to be treated are mixed resulting a solid loading of about 0-90%. The reaction conditions are about 0-160° C., about 0-500 Bar, and the reaction time varies from about 1 minute to about 2 days, in batch, semi-batch or continuous mode.
2) The resulting pretreated solids are separated from the liquids. The pretreated solids have higher enzymatic digestibility and the resulting hemicellulose hydrolysate contains less inhibitor formation compared to conventional (e.g., sulfuric acid pretreatment methods).
3) The process does not require the addition of exogenous chemicals.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. REFERENCES

1. Alvarez, H. M., et al. (1996) Formation of intracytoplasmic lipid inclusions by Rhodococcus opacus strain PD630, Arch Microbiol, 165, 377-386.
2. Humbird, D., et al. (2011) Process Design and Economics for Biochemical Conversion of Lignocellulosic Biomass to Ethanol Dilute-Acid Pretreatment and Enzymatic Hydrolysis of Corn Stover, NREL Report No. TP-5100-47764 Golden, Colorado; NREL (National Renewable Energy Laboratory)
3. Lin, H., et al. (2017) Engineering Neurospora crassa for cellobionate production directly from cellulose without any enzyme addition, Enzyme Microb Technol, 99, 25-31.
4. Lin, Y. J., Hestekin, J. A. and Sather, N. (2016) Bioprocessing of Cost-Competitive Biobased Organic Acids. In, Commercializing Biobased Products: Opportunities, Challenges, Benefits, and Risks. The Royal Society of Chemistry, Green Chemsitry Series No. 43.
5. Lu, Y. L. and Mosier, N. S. (2008) Kinetic Modeling Analysis of Maleic Acid-Catalyzed Hemicellulose Hydrolysis in Corn Stover, Biotechnol. Bioeng., 101, 1170-1181.
6. Mosier, N., et al. (2005) Features of promising technologies for pretreatment of lignocellulosic biomass, Bioresour Technol, 96, 673-686.
7. Mosier, N. S., Ladisch, C. M. and Ladisch, M. R. (2002) Characterization of acid catalytic domains for cellulose hydrolysis and glucose degradation, Biotechnol. Bioeng., 79, 610-618.
8. Wyman, C. E., et al. (2005) Comparative sugar recovery data from laboratory scale application of leading pretreatment technologies to corn stover, Bioresour. Technol., 96, 2026-2032.
9. Wyman, C. E., et al. (2005) Coordinated development of leading biomass pretreatment technologies, Bioresour. Technol., 96, 1959-1966.
10. Yang, B., et al. (2011) Enzymatic hydrolysis of cellulsic biomass, Biofuels, 2, 421-450.
11. Donaldson, L. A., Wong, K. K. Y. and Mackie, K. L. (1988) Ultrastructure of Steam-Exploded Wood, Wood Sci Technol, 22, 103-114.
12. Lynd, L. R., Wyman, C. E. and Gerngross, T. U. (1999) Biocommodity Engineering, Biotechnol. Prog., 15, 777-793.

1. A method for producing a fuel or a commodity chemical from a cellulosic biomass, the method comprising:
(a) pretreating a composition comprising the cellulosic biomass with a sugar acid, thereby producing a pretreated composition;
(b) fermenting at least some of the pretreated composition in a first fermentation process, thereby producing a first fermented composition, wherein the first fermented composition comprises the sugar acid;
(c) separating the first fermented composition into a first fraction and a second fraction, wherein the first fraction comprises at least some of the sugar acid and the second fraction comprises at least some of the sugar acid; and
(d) fermenting the second fraction isolated in step (c) in a second fermentation process, thereby producing the fuel and/or commodity chemical.

2. The method of claim 1, wherein the sugar acid comprises an oligosaccharide aldonic acid, a disaccharide aldonic acid, a monosaccharide aldonic acid, a heteropolysaccharide aldonic acid, or a combination thereof; and/or
wherein the sugar acid is selected from the group consisting of cellobionic acid (CBA), gluconic acid (GA), glucuronic acid, xylonic acid, glucaric acid, and a combination thereof.

3. (canceled)

4. The method of claim 1, wherein at least some of the sugar acid present in the first fraction separated in step (c) is used in subsequent cellulosic biomass pretreatment.

5. The method of claim 1, wherein the sugar acid is present at a concentration of up to about 99% by volume during the pretreatment in step (a).

6. (canceled)

7. The method of claim 1, wherein the composition being pretreated in step (a) comprises up to about 90% solids by volume.

8. (canceled)

9. The method of claim 1, wherein the pretreatment in step (a) is performed at a temperature of about 0° C. to about 220° C.; and/or
wherein the pretreatment in step (a) is performed at a pressure of about 0 bar to about 500 bar; and/or
wherein the pretreatment in step (a) is performed in batch, semi-batch, or continuous mode.

10-12. (canceled)

13. The method of claim 1, wherein the composition is pretreated in step (a) for about 1 minute to about 2 days.

14. (canceled)

15. (canceled)

16. The method of claim 1, wherein the pretreated composition produced in step (a) is separated into a first phase that predominantly comprises liquids and a second phase that predominantly comprises solids.

17. The method of claim 16, wherein the first phase comprises hemicellulose hydrolysate, GA, and/or glucose, and wherein at least some of the hemicellulose hydrolysate, GA, and/or glucose are used in the second fermentation process in step (d); and/or
wherein the second phase comprises pretreated cellulosic biomass, and wherein at least some of the pretreated cellulosic biomass is converted to the sugar acid by an engineered host cell during the first fermentation process in step (b).

18. (canceled)

19. (canceled)

20. The method of claim 1, wherein the first fermentation process in step (b) comprises an aerobic fermentation process.

21. (canceled)

22. (canceled)

23. The method of claim 17, wherein the engineered host cell comprises:
(a) reduced activity of one or more polypeptides having β-glucosidase activity as compared to a corresponding wild-type cell, wherein each of the one or more polypeptides having β-glucosidase activity are encoded by a gene that has at least about 80% sequence identity to a gene selected from the group consisting of NCU00130, NCU04952, NCU05577, NCU07487, NCU08755, and NCU03641;
(b) reduced activity of a polypeptide having cellobionate phosphorylase activity as compared to a corresponding wild-type cell, wherein the polypeptide having cellobionate phosphorylase activity is encoded by a gene that has at about least 80% sequence identity to NCU09425 (NdvB);
(c) reduced activity of a polypeptide encoded by a gene that has at least about 80% sequence identity to NCU08807 (CRE-1) as compared to a corresponding wild-type cell;
(d) reduced activity of a polypeptide encoded by a gene that has at least about 80% sequence identity to NCU09333 (ACE-1) as compared to a corresponding wild-type host cell; and/or
(e) an increased expression or activity of a laccase protein as compared to a corresponding wild-type cell.

24. (canceled)

25. The method of claim 23, wherein the engineered host cell is cultured in a media that contains cycloheximide to increase the expression or activity of the laccase protein.

26. The method of claim 1, wherein the separation performed in step (c) comprises using electrodeionization (EDI).

27. The method of claim 1, wherein pretreatment of the cellulosic biomass is performed at a lower temperature compared to when the sugar acid is not used to pretreat the cellulosic biomass.

28. The method of claim 1, wherein the glucose yield of the pretreatment in step (a) is higher compared to when the sugar acid is not used to pretreat the cellulosic biomass.

29. The method of claim 1, wherein the glucose yield of the pretreatment in step (a) is at least about 75% to about 95%.

30. The method of claim 1, wherein the amount of cellulase inhibitory compounds that are produced during the pretreatment in step (a) is lower compared to when the sugar acid is not used to pretreat the cellulosic biomass.

31. The method of claim 1, wherein the amount of hemicellulose and/or lignin that is removed from the cellulosic biomass during the pretreatment in step (a) is higher compared to when the sugar acid is not used to pretreat the cellulosic biomass.

32. A fuel and/or a commodity chemical produced by the method of claim 1.

33. A method for producing gluconic acid (GA), the method comprising separating hemicellulose hydrolysate by electrodeionization (EDI).
